(19) United States
(12) Patent Application Publication
(10) Pub. No.: US 2025/0001202 A1
(43) Pub. Date: Jan. 2, 2025

(54) THE METHOD OF SIMULTANEOUSLY IMAGING THE DOSING AMOUNT AND PROVIDING FEEDBACK IN PHOTODYNAMIC APPLICATIONS

(71) Applicant: GEBZE TEKNIK UNIVERSITESI, Kocaeli (TR)

(72) Inventors: Kamil CINAR, Istanbul (TR); Asiye YURTTAS, Istanbul (TR)

(73) Assignee: GEBZE TEKNIK UNIVERSITESI, Kocaeli (TR)

(21) Appl. No.: 18/696,418

(22) PCT Filed: Feb. 21, 2022
(86) PCT No.: PCT/TR2022/050153

§ 371 Date: Mar. 28, 2024

(30) Foreign Application Priority Data
Sep. 28, 2021 (TR) 2021/015110

(51) Int. Cl. A61N 5/06; A61N 5/067

(52) U.S. Cl. CPC A61N 5/062; A61N 5/067; A61N 2005/0627; A61N 2005/066

(57) ABSTRACT

A method that simultaneously measures how effective the photodynamic application and/or therapy irradiation amounts and the therapy efficiency are on the unit cell-organism or in cell-microorganism communities is provided. The method uses a feedback mechanism without the need for an operator during therapy in cases where dosing is insufficient or excessive.

### CROSS REFERENCE TO THE RELATED APPLICATIONS

This application is the national phase entry of International Application No. PCT/TR2022/050153, filed on Feb. 21, 2022, which is based upon and claims priority to Turkish Patent Application No. 2021/015110, filed on Sep. 28, 2021, the entire contents of which are incorporated herein by reference.

### TECHNICAL FIELD

The invention relates to a method for use in in vitro and/or in vivo studies in hospitals and/or laboratories, that measures simultaneously how effective the photodynamic application and/or therapy irradiation (dosing) amounts and the therapy efficiency are on the unit cell-microorganism (cells, bacteria, fungi, etc.) in the systems (animals, humans, plants, etc.) or in cell-microorganism (cells, bacteria, fungi, etc.) communities and that controls the dose amount by its own feedback mechanism without the need for any operator during therapy in cases where dosing is insufficient or excessive.

### BACKGROUND

In current photodynamic therapy applications, the light dose amounts applied in in vivo or in vitro studies and how effective the photosensitizer is in which living organism, and the vitality rates after the therapy are determined according to the data obtained as a result of many cytotoxicity tests and animal experiments. It is administered in a different living organism with reference to articles or with reference to these predetermined dose amounts to be administered to the patient. Because of the environmental conditions of living organisms and the large number of factors they contain within themselves, dosing and intensity amounts cause a difference in the effectiveness of the doses and photosensitizers determined in the previous experiments, it is necessary to change the intensities of dosing and photosensitizers in order to optimize them repeatedly for each subject/patient/cell/sample. These trial-and-error adjustments (optimization) lead to losses arising from the use of time, labor and materials in the health sector or laboratory environments of a commercialized product. It can be said to give an example on a cell basis that after the cell is given photosensitizer at certain concentrations and time, cytotoxicity tests are performed to calculate the % viability rates and to conduct statistical studies and to graph it, and the costs of the imported materials used lead to the loss of national income.

Various studies were carried out in the art on the realization of dosing adjustment in photodynamic applications.

The United States patent document numbered US2014171927A1, which is in the state of the art, mentions an improved method for the treatment of cornea and/or sclera in an eye to correct a laser therapy system and a refractive system. The relevant document does not describe a method for measuring how effective the photodynamic administration and/or therapy irradiation (dosing) amounts, and simultaneous dosing is in the unit-organism, and for providing feedback on the dose amount during therapy in cases where dosing is insufficient or excessive.

A laser nano-optic diagnostic and treatment device is mentioned in the Chinese patent document numbered CN104398238A, which is in the state of the art. The relevant device allows non-invasive tumor treatment on the treatment object by monitoring the metabolism, distribution and enrichment of the nano-light sensitizer in real time and positioning the tumors correctly.

The Chinese patent document numbered CN110681070A, which is in the state of the art, mentions a photodynamic therapy light source and an editing method that can make personalized edits. The laser light source is mentioned in the relevant method.

In the United States patent document numbered US2011238002A1 (D4), which is in the state of the art, a device developed for the photo-dynamic therapy of living organism tissues is mentioned.

When the methods described in the art are examined, there is a need for developing a method that measures simultaneously how effective the photodynamic application and/or therapy irradiation (dosing) amounts and the therapy efficiency are on the unit cell-microorganism (cells, bacteria, fungi, etc.) in the systems (animals, humans, plants, etc.) or in cell-microorganism (cells, bacteria, fungi, etc.) communities and that controls the dose amount by its own feedback mechanism without the need for any operator during therapy in cases where dosing is insufficient or excessive.

### SUMMARY

The object of this invention is to carry out a method that measures simultaneously how effective the photodynamic application and/or therapy irradiation (dosing) amounts, and the therapy efficiency are on the unit cell-microorganism (cells, bacteria, fungi, etc.) in the systems (animals, humans, plants, etc.) or in cell-microorganism (cells, bacteria, fungi, etc.) communities.

Another object of the present invention is to carry out a method that controls the dose amount by its own feedback mechanism without the need for any operator during therapy in cases where dosing is insufficient or excessive in photodynamic applications.

Another object of the present invention is to stimulate and terminate the viability of the target organism simultaneously with the wavelength of laser light associated with the active photosensitizer in vitro or in vivo and to realize a method that enables simultaneous measurement and imaging of % viability rates without the need for cytotoxicity tests.

Another object of the present invention is to carry out a method that simultaneously ensures that the laser fluence amounts are brought to the required level during the therapy application if the relevant laser radiation is not effective in the target organism during photodynamic therapy and/or its effectiveness does not reduce the desired level of viability.

### DETAILED DESCRIPTION OF THE EMBODIMENTS

The method of the invention includes the following steps;

Determining the killing dose (or laser parameters) of the laser to be used in the application within non-target (desired to be protected in healthy and living organisms) units to non-target units in the application,
ensuring that each unit absorbs photosensitizer by giving photosensitizer to target/non-target units in the same environment,
then determining the minimum time (Tₛ) required for them to remove from their content,
after the Tₛ period, exposing the target units to the laser fluence (Φ) for the determined dosing time (t_{d}), provided that it is primarily observed,
giving photosensitizer to the application environment by preparing the target/non-target units to be applied again,
waiting for Tₛ time and taking microscopic scale images with the imaging system by dividing the difference between the maximum permissible fluence and the minimum active fluence Φ_{M}−Φₘ) with the laser in the relevant wavelength (NIR/SWIR) in the unit area by 10 or more integer numbers,
   applying the photosensitizer to destroy the target tissue, parasite or organism determined by the expert in the target/non-target organism to be applied the photodynamic therapy/application in a healthy body,
waiting for Tₛ time and ensuring that healthy (non-target cells/units) remove photosensitizer from their bodies,
positioning the imaging and application laser ports in the relevant target area,
focusing optical systems for microscopic image,
the laser provides both the illumination of the imaging system and the radiation of the photodynamic therapy application at the same time,
applying the relevant photosensitizer with the most absorbent NIR/SWIR wavelength laser application by entering the Φ_{M}, t_{d} determined for the relevant target organism in the database,
during the application, the microscopic image acquisition process focused on the relevant area is simultaneously taken with 1 s-5 s time periods,
with Machine Learning, counting of CH, MH, ChO, and MhO numbers at determined periods,
calculating the CH/MH, MhO/ChO, MhO/MH ratios after the count,
if the machine learning during photodynamic therapy detects a decrease from the application starting values at the ChO/MhO ratio obtained from consecutive images, the laser fluence decreases the Φᵤ value in the ΔΦ stages and brings the Φᵤ value up to the Φₘ value, and
if the decrease in the ChO/MhO ratio continues, terminating the system's laser fluence and photodynamic therapy/application.

In the method of the invention, the efficiency of the light intensity sent to the application site in the unit area and/or unit time by the light sources (LED, halogen lamp, laser, etc.) used in photodynamic therapy (PDT) and/or applications in the target building blocks such as unhealthy parasitic cancerous cells or tissues and the efficiency thereof in the healthy non-target living building blocks are quantitatively determined by counting the quantity of subunits (or building blocks) such as cells, bacteria, viruses, etc.

The cell, unit, building block, unit cell-microorganism described in the method of the invention are defined as the unit cell-microorganism (cell, bacteria, fungi, etc.) that forms systems (animals, humans, plants, etc.) and/or communities and/or tissues.

As a result of the calculation of certain ratios by processing the snapshots taken during the application, the laser radiation parameters are controlled with feedback. In addition, the quantitative determination of unhealthy parasitic cancerous cells simultaneously during therapy will be gained from both time and cost without the need for viability tests. Viability tests require a long-term laboratory process. Classical viability tests carried out within 3-4 days with the method of the invention will be carried out within seconds. The method of the invention will determine the viability rates of unhealthy parasitic cancerous cells etc. simultaneously during therapy/application.

The light sources used in the method are narrow frequency bands and preferably laser sources. These light sources are pulsed light sources that can radiate in near-infrared (NIR: 750 nm-1400 nm) and/or short wavelength infrared (SWIR: 1400 nm-3000 nm) optical windows and operate in microsecond (μs) to picosecond (ps) intervals.

The imaging system used in the method includes longpass and/or NIR/SWIR bandpass filter(s) with optical permeability to include the bandwidth of the applied laser source of the system, consisting of one or more optical lenses and/or lens sets with a magnification that can collect completely microscopic scale images from the targeted area and distinguish unhealthy parasite bacteria cancerous cell etc. building blocks, to distinguish structures ranging between 100 nm-1 mm sizes.

The light intensity adjustment feedback mechanism used in the method of the invention enables the number of unhealthy parasitic bacterial cancerous cells, etc. and non-target healthy cell groups, which are the related targets, in the microscopic area and/or volume to be counted and compared on the microscopic scale images of the structures in the target area. In addition, it is ensured that the amounts of photosensitizers in the contents of the target unhealthy parasite bacteria cancerous cells etc. and non-target healthy cell groups pass/reflect the radiation at the above-mentioned NIR/SWIR wavelengths and the shapes, sizes and contrast rates of the structures forming the groups are determined.

The method of the invention is a simultaneous feedback mechanism using pulsed laser source operating in a kind of NIR or SWIR optical spectrum, which supports itself with machine learning. The feedback mechanism obtains the necessary data by counting the distinguishable quantity of the target/non-target building blocks from the target region. The smallest units that make up the target and/or non-target organisms, which are meant by the building blocks or units mentioned, refer to bacteria, viruses, cells, etc.

In the method of the invention, the criteria and technical requirements used for machine learning consist of two parts as before and during the application/therapy as described below. Pre-application/therapy provides data sets for machine learning for the feedback mechanism of the technical system, while it is essential for more precise determination of the relevant harmful target structures during application/therapy and counting on the basis of building blocks.
1. Determining the killing dose (or laser parameters) of the laser to be used in the application within non-target (desired to be protected in healthy and living organisms) units to non-target units in the application: In this step, no photosensitizer is used and only the units are exposed to laser. The maximum laser parameters (fluence and application/dosing time) are determined according to the wavelength of the application laser, the pulse parameters, and the amount of fluence that the non-target healthy unit can maintain its viability. Here the pulse length and the wavelength of the laser are kept constant. The determined dosing time (t_{d}) and the amount of fluence (Φ_{M}) are recorded as maximum laser parameter limits to be used in the data set required for machine learning in the clinical and/or laboratory environment. t_{d} and Φₘ are limit values and these values will not be exceeded during application.
2. After each unit absorbs photosensitizer by giving photosensitizer to target/non-target units in the same environment, the minimum time (Tₛ) required for them to remove from their content is determined. Here, it is ensured that more photosensitizers remain in the target units during the time that passes enough for a minimum amount of photosensitizer to remain in the content of the important non-target units. The Tₛ time usually varies between 2-12 hours and depends on the photosensitizer used in the application. It should be optimized with clinical and/or laboratory experiments to be performed before the application for the method. The method herein applies to general photodynamic applications and is not provided for a particular photosensitizer.
3. After the Tₛ time, the target units are exposed to the laser fluence (Φ) for the determined dosing time (t_{d}), provided that it is primarily observed. The aim at this stage is to bring the laser light to the minimum amount of fluence and to ensure the death/termination of the target units during ta with the minimum fluence (Φm).
4. The target/non-target units to be applied are prepared again and photosensitizer is given to the application environment. The Tₛ time is waited for. Microscopic scale images are taken with the imaging system by dividing the difference between the maximum permissible fluence and the minimum active fluence (Φ_{M}−Φₘ) with the laser in the relevant infrared wavelength (NIR/SWIR) in the unit area by 10 or more integer numbers.

The maximum permissible fluence and minimum active fluence amounts determined experimentally shall be determined, recorded, and kept in the device by preliminary experiments for the photosensitizer used in the application and the organism and/or organism building blocks to be applied on it and the harmful target (organism building blocks/tissue planned to be destroyed in the application) organism structures.

### Example

$$ΔΦ = ( {Φ}_{M} - {Φ}_{m} ) / 20 \qquad ( Formula I )$$

ΔΦ: Fluence level
a. In the images taken, the dimensions and shapes of the target/non-target units are entered into the machine learning program by default and the numbers of the units are counted by considering their shape, size and contrasts under the relevant light source.
b. The images taken at each fluence level (ΔΦ) increase are counted by entering them again in the machine learning database by looking at this target/non-target unit shapes, dimensions and contrast ratios. The numbers to be determined here and the difference to be examined are calculated depending on the amount of fluence.

$$Φ = n \cdot ΔΦ + {Φ}_{m} \qquad ( Formula II )$$

The amount of fluence (Φ=n·ΔΦ+Φₘ) consists of 5 data sets with the numbers of units of the following definitions:
If the amount of fluence=Φ, in the unit area or volume,
Number of Living Target Units: CH,
Number of Non-Living Target Units: MH,
Number of Living Non-Target Units: ChO,
Number of Non-Living Non-Target Units: MhO.
   c. Machine learning of living units and non-living units distinguishes the amount of reflection and absorption of the relevant target and non-target units at the shape and relevant wavelength and the amount of contrast between living units and non-living units. The shape definitions here will differ for the building blocks of the target/non-target organisms that can be mentioned in each case and should be defined in advance during the creation of the relevant database. These definitions are made during the clinical and/or laboratory preliminary studies phase and then entered into the database of machine learning for application.
   d. This machine learning software calculates the CH/MH, MhO/ChO, MhO/MH ratios in Φ value for the laser and photosensitizer used in the application by comparing the shape, contrast and size of the large number of images taken to determine the above CH, MH, ChO and MhO numbers.
   e. This machine learning software determines the closest laser application fluence (Pu) to CH/MH≈0, MhO/ChO≈0, MhO/MH≈0 for the predetermined photosensitizer used in the application.

The values determined above are recorded in the database for photodynamic therapy/target/non-target unit pairs to be applied.
5. Photodynamic therapy/application of photosensitizer is applied to destroy the target tissue, parasite or organism determined by the expert in the target/non-target organism to be applied in a healthy body. Tₛ duration specified in Article 2 is waited and healthy (non-target) cells/units are ensured to remove photosensitizer from their bodies. The imaging and application laser ports are positioned in the relevant target area. Optical systems focus for microscopic image. The laser provides both the illumination of the imaging system and the radiation of the photodynamic therapy application at the same time. During focusing and initial image acquisition, the laser is operated at a power corresponding to the fluence below 80% of the specified Φₘ value during therapy/application. This is only used for focusing and first-hand image acquisition. After the focusing process is finished, the system parameters are adjusted to work on their own as described in the following in the image acquisition. The system does not need an external illuminator even to take an image in this way. By entering the Φ_{M}, t_{d}, determined for the relevant target organism in the database in Article 4, the most absorbent NIR/SWIR wavelength laser application of the relevant photosensitizer is initiated. The application starts with the application of the laser fluence Φₘ value. During the application, the microscopic image acquisition process focused on the relevant area is simultaneously taken with 1 s-5 s time periods. With Machine Learning, the CH, MH, ChO and MhO numbers specified in Article 4b perform the counts at determined periods and calculate the CH/MH, MhO/ChO, MhO/MH ratios. Laser fluence increases simultaneously to bring the CH/MH, MhO/ChO, MhO/MH ratios specified in Article 4d closer to the “0” value by comparing each consecutive image in the Machine Learning database and/or taken during the application. If the ratios of CH/MH, MhO/ChO, MhO/MH are moving away from 0, the value of Φᵤ is increased by the steps of ΔΦ. In practice, if the values are close to zero, the system is ensured to operate for the ta time in the eyes of the expert operator, during which time laser irradiation is continued.
6. If the machine learning during photodynamic therapy detects a decrease from the application starting values at the ChO/MhO ratio obtained from consecutive images, the laser fluence can reduce the Φᵤ value in the AP stages and bring the Φᵤ value up to the Φₘ value. If the decrease in the ChO/MhO ratio continues, the system terminates the laser fluence and photodynamic therapy/application is terminated.
7. During the image collection, while the machine learning code performs formal and dimensional unit counts using the image recognition algorithm, brightness, contrast, gamma optimization algorithms are used for target/non-target and living/non-living comparisons.

What is claimed is:

1. A method for simultaneously measuring an effective dosing amount and a therapy efficiency on a unit-organism in an organism in which dosing is performed in photodynamic applications, wherein the method provides feedback on a dose amount during therapy in cases where dosing is insufficient or excessive, the method comprising the following steps;
determining parameters of a laser to be used in a photodynamic application within target units compared to non-target units,
ensuring that each unit absorbs a photosensitizer by giving the photosensitizer to the target units and the non-target units in the same environment,
determining the minimum time (Tₛ) required for the target units and the non-target units to remove from their content,
after time Tₛ, subjecting the target units to the laser fluence (Φ) for the determined dosing time (t_{d}),
waiting for time Tₛ and taking microscopic scale images with an imaging system by dividing the difference between the maximum permissible fluence and the minimum active fluence (Φ_{M}−Φₘ) with the laser in the unit area wavelength (NIR/SWIR) by 10 or more whole numbers,
applying the photosensitizer to a target organism and a non-target organism,
waiting for time Tₛ and ensuring that healthy non-target units remove the photosensitizer,
positioning the imaging and application laser ports in the relevant target area,
focusing optical systems for microscopic image,
applying the photosensitizer with the most absorbent NIR/SWIR wavelength laser application by entering the Φ_{M}, t_{d} values determined for the target organism in the database,
during the laser application, simultaneously taking microscopic images focused on the relevant area in 1 s-5 s time periods,
performing counts of a number of living target units (CH), a number of non-living target units (MH), a number of living non-target units (ChO), and a number of non-living non-target units (MhO) with machine learning,
calculating a ratio of the number of living target units and the number of non-living target units (CH/MH), a ratio of the number of non-living non-target units and the number of living non-target units (MhO/ChO), and a ratio of the number of non-living non-target units and the number of non-living target units (MhO/MH) after the count,
decreasing the laser fluence Φᵤ value in the ΔΦ stages and bringing the Φᵤ value up to the Φₘ value when a decrease is detected from the application starting values in the ratio of the number of living non-target units and the number of non-living non-target units (ChO/MhO) obtained,
terminating the laser fluence when the decrease in the ratio of the number of living non-target units and the number of non-living non-target units (ChO/MhO) continues.

2. The method according to claim 1, wherein a pulse length and the wavelength of the laser are kept constant during the step of determining the laser parameters of the laser to be used in the application within the target units to the non-target units in the application.

3. The method according to claim 1, wherein it is ensured that more photosensitizers remain in the target units within a period until the minimum amount of photosensitizer remains in the content of the non-target units.

4. The method according to claim 1, wherein during focusing and initial image acquisition, the laser is operated at a power corresponding to the fluence below 80% of the Φₘ value determined.

5. The method according to claim 1, wherein the wavelength used in the method is between 750 nm-3000 nm.

6. The method according to claim 1, wherein the time Tₛ value used in the method is between 2 and 12 hours.
